(19) United States
(12) Patent Application Publication
(10) Pub. No.: US 2019/0350258 A1
(43) Pub. Date: Nov. 21, 2019

(54) ATOMIZATION CORE AND ATOMIZER THEREOF

(71) Applicant: SHENZHEN UWELL TECHNOLOGY CO., LTD., Shenzhen (CN)

(72) Inventor: YUCHENG XIAO, Shenzhen (CN)

(21) Appl. No.: 16/027,270

(22) Filed: Jul. 4, 2018

(30) Foreign Application Priority Data
May 15, 2018 (CN) 2018207184654

(51) Int. Cl. A24F 47/00

(52) U.S. Cl. CPC A24F 47/008; A61M 11/042

(57) ABSTRACT

An atomization core includes a housing, a hollow frame to form a gas passage therein, a heating wire with two opposite ends thereof respectively connected to a first polarity pin and a second polarity pin, an organic cotton surrounded around the heating wire and received in the gas passage, an insulating portion mounted on a bottom of the gas passage and spaced from a bottom of the organic cotton, and an electrode post mounted on the insulating portion and insulated from the frame. The housing is covered on the frame with an air outlet thereof. The frame includes an air inlet and a connecting portion respectively formed at the bottom thereof to connect to a battery. The first polarity pin extends along the bottom of the gas passage through the insulating portion and connects to the electrode post, the second polarity pin connecting to an inner wall of the frame.

### BACKGROUND

### 1. Technical Field

The present disclosure generally relates to electronic cigarettes field, and especially relates to an atomization core and an atomizer thereof.

### 2. Description of Related Art

A conventional atomizer of an electronic cigarette includes a mouthpiece assembly for suction gas, an e-liquid storage assembly for storing tobacco liquid therein, an atomization core for vaporizing the tobacco liquid and a base assembly.

A positive electrode pin or a negative electrode pin of the conventional atomization core is indirectly and electrically connected to a battery assembly by means of a corresponding electrode conducting element mounted on the base assembly so that a contact resistance is increased after completing assembly of the atomization core. At the same time, since the electrode conducting element is subjected to deformation in the case of thermal expansion and contraction so that a resistance value of the atomization core is easily changed because its deformation under a hot condition, which can minimize the lifespan of the atomization core.

Therefore, a new atomization core and an atomizer thereof should be developed in order to solve the problems shown above.

### SUMMARY

The technical problems to be solved: in view of the shortcomings of the related art, the present disclosure relates to an atomization core and an atomizer thereof which can solve the problem that a conventional atomization core has a large contact resistance during usage, easy to change the resistance value, and easy to cause the atomization core to become hot so as to affect the lifespan of the electronic cigarette.

The technical solution adopted for solving technical problems of the present disclosure is:
an atomization core includes a cylindrical hollow frame including a gas passage formed along its axis direction thereof, an air inlet formed on a sidewall at a bottom thereof to connect with the gas passage, and a connecting portion formed at a lower portion thereof near the air inlet and directly connected to a battery assembly; a housing covered on the frame and including an air outlet formed close to an upper end of the gas passage; a heating wire including two opposite ends respectively connected to a first polarity pin and a second polarity pin; an organic cotton surrounded around the heating wire and received in the gas passage; an insulating portion mounted on a bottom of the gas passage and insulated isolation from a bottom of the organic cotton; an electrode post mounted on the insulating portion and insulated from the frame. Each e-liquid inlet is respectively formed at a sidewall of the housing and a sidewall of the frame to connect to the organic cotton, and the first polarity pin is extended along the bottom of the gas passage through the insulating portion and connected to the electrode post, the second polarity pin is connected to an inner wall of the frame.

Wherein the insulating portion includes an insulating gasket and an electrode gasket mounted in sequence on the bottom of the gas passage, with an upper portion of the insulating gasket separating from the organic cotton and a lower portion attaching to the electrode gasket; the insulating gasket including an installing hole and a first through-hole formed at interval along an axial direction of the insulating gasket, the first through-hole connected with the installing hole at a bottom of the insulating gasket; the electrode gasket including a second through-hole along its axis direction thereof, one end of the second through-hole enlarged to form a sink hole and the other opposite end of the second through-hole connected to the installing hole; the electrode post including a rod portion passing through the second through-hole and the installing hole, and a base portion mounted on the sink hole; and the first polarity pin received in the first through-hole and then connected to the electrode post after bending its upper portion above an upper end of the electrode gasket.

Wherein the insulating gasket further includes a connecting recess formed at a bottom thereof to connect to the first through-hole and the installing hole, the first polarity pin connected to the rod portion after bending it.

Wherein the frame further includes a hollow upper frame, a hollow lower frame and an insulating sleeve formed between the upper frame and the lower frame, the gas passage passing through the upper frame and the lower frame. The insulating sleeve includes a connecting hole formed in a middle portion thereof to connect the gas passage of the upper and lower frames, a bottom portion of the upper frame extending into the gas passage of the lower frame, the housing sleeved on the upper frame, the e-liquid inlet arranged on the housing and a sidewall of the upper frame, the air inlet arranged on a sidewall of the lower frame and the connecting portion arranged on a lower end of the air inlet of the lower frame; both the organic cotton and the heating wire received in the gas passage of the upper frame; the insulating portion and the electrode post respectively mounted on a bottom portion of the lower frame to seal the bottom portion of the lower frame; the first polarity pin passing through the connecting hole of the insulating sleeve and the second polarity pin pressed between an outer wall of the insulating sleeve and an inner wall of the upper frame and connected to the inner wall of the upper frame.

Wherein the upper frame further includes a first stepping surface formed at an upper portion thereof, and a second stepping surface formed at a lower portion thereof; an upper portion of the insulating sleeve inserting into the gas passage of the upper frame, with a bottom surface of the insulating sleeve abutting against the first stepping surface; the insulating gasket received in the gas passage of the lower frame and abutted against the second stepping surface, the bottom surface of the insulating sleeve separated from a top surface of the insulating gasket.

Wherein the insulating sleeve further includes an installing groove formed at the outer wall of the bottom thereof, the second polarity pin pressed against the outer wall of the insulating sleeve and the inner wall of the upper frame is contacted with an inner wall of the lower frame after bending it in the installing groove.

Wherein both the insulating portion and the insulating sleeve are made of high temperature resistant plastic material.

Wherein the connecting portion is a thread arranged in a ring direction on the outer wall of the lower frame.

Wherein the frame is a hollow integral structure and includes an upper frame portion and a lower frame portion, the housing sleeved on the upper frame portion, the e-liquid inlet arranged on the housing and a sidewall of the upper frame portion, the air inlet arranged on a sidewall of the lower frame portion and the connecting portion arranged on a lower end of the air inlet of the lower frame portion; the organic cotton and the heating wire respectively received in the gas passage of the upper frame portion; the insulating portion and the electrode post respectively mounted on a bottom portion of the lower frame portion to seal the bottom portion of the lower frame portion; the first polarity pin passing through the lower frame portion and connected to the electrode post, the second polarity pin passing through the lower frame portion and pressed between an outer wall of the insulating gasket and an inner wall of the lower frame portion and connected to the inner wall of the lower frame portion.

An atomizer according to an exemplary embodiment of the present disclosure includes a hollow mouthpiece assembly, a cover member, an e-liquid storage assembly, a base assembly and an atomization core in sequential connection. The atomization core includes a cylindrical hollow frame including a gas passage formed along its axis direction thereof, an air inlet formed on a sidewall at a bottom thereof to connect with the gas passage, and a connecting portion formed at a lower portion thereof near the air inlet and directly connected to a battery assembly; a housing covered on the frame and including an air outlet formed close to an upper end of the gas passage; a heating wire including two opposite ends respectively connected to a first polarity pin and a second polarity pin; an organic cotton surrounded around the heating wire and received in the gas passage; an insulating portion mounted on a bottom of the gas passage and insulated isolation from a bottom of the organic cotton; an electrode post mounted on the insulating portion and insulated from the frame. Each e-liquid inlet is respectively formed at a sidewall of the housing and a sidewall of the frame to connect to the organic cotton, and the first polarity pin is extended along the bottom of the gas passage through the insulating portion and connected to the electrode post, the second polarity pin is connected to an inner wall of the frame. The housing and one part of the frame are received in the e-liquid storage assembly and the other part of the frame is received in the base assembly. The connecting portion is extended out of the base assembly to connect to the battery assembly. The base assembly includes a gas inlet formed on a sidewall thereof to connect to the air inlet of the frame, and the mouthpiece assembly includes a gas vent so that gas can enter the gas passage of the atomization core from the gas inlet of the base assembly and then is discharged out from the gas vent of the mouthpiece assembly.

The present disclosure provides the advantages as below.

The structure of the present disclosure is provided the below configuration: the insulating portion and the electrode post are arranged on the bottom of the frame so that the electrode post is separated from the frame by the insulating portion; the organic cotton and the heating wire are received in the gas passage of the frame, the first polarity pin of the heating wire passing through the insulating portion to connect to the electrode post and the second polarity pin connected to the inner wall of the frame. A connecting portion is formed on the lower portion of the frame. In this way, the atomization core can directly connect to the battery assembly by the connecting portion, the first polarity pin can directly and electrically connect to a battery of the battery assembly by the electrode post, thereby the contact resistance can be reduced by reducing indirect contact of intermediate conductors. At the same time, no indirect conduction of intermediate conductors can also prevent the pulse of the resistance value. The atomization core can't become too hot because the contact resistance is too large or the resistance value jumps so that the lifespan of the atomization core can be improved.

### BRIEF DESCRIPTION OF THE DRAWINGS

Many aspects of the embodiments can be better understood with reference to the following drawings. The components in the drawings are not necessarily dawns to scale, the emphasis instead being placed upon clearly illustrating the principles of the embodiments. Moreover, in the drawings, like reference numerals designate corresponding parts throughout the several views.

FIG. 1 is a schematic view of the atomization core in accordance with a first exemplary embodiment.

FIG. 2 is a cross-sectional schematic view of the atomization core of FIG. 1.

FIG. 3 is an exploded, schematic view of the atomization core of FIG. 1.

FIG. 4 is a schematic view of an insulating gasket of the atomization core of FIG. 3.

FIG. 5 is similar to FIG. 4, but shown the insulating gasket inverted.

FIG. 6 is a cross-sectional schematic view of an electrode gasket of the atomization core of FIG. 3.

FIG. 7 is a schematic view of a lower frame of the atomization core of FIG. 3.

FIG. 8 is a cross-sectional schematic view of the lower frame of the atomization core of FIG. 7.

FIG. 9 is a schematic view of an insulating sleeve of the atomization core of FIG. 3.

FIG. 10 is a cross-sectional schematic view of the atomization core in accordance with a second exemplary embodiment.

FIG. 11 is an exploded, cross-sectional schematic view of the atomization core of FIG. 10.

FIG. 12 is a cross-sectional schematic view of the atomizer in accordance with the first exemplary embodiment.

### DETAILED DESCRIPTION

It will be appreciated that for simplicity and clarity of illustration, where appropriate, reference numerals have been repeated among the different figures to indicate corresponding or analogous elements. In addition, numerous specific details are set forth in order to provide a thorough understanding of the embodiments described herein. However, it will be understood by those of ordinary skill in the art that the embodiments described herein can be practiced without these specific details. In other instances, methods, procedures and components have not been described in detail so as not to obscure the related relevant feature being described. Also, the description is not to be considered as limiting the scope of the embodiments described herein. The drawings are not necessarily to scale and the proportions of certain parts have been exaggerated to better illustrate details and features of the present disclosure. The disclosure is illustrated by way of example and not by way of limitation in the figures of the accompanying drawings, in which like reference numerals indicate similar elements.

In the description of the present disclosure, it needs to be explained that all the directional indicators (such as the terms: “upper”, “below”, “left”, “right”, “front”, “back” . . . ), are shown in the specification of the present disclosure. The indicated orientation or position of the terms shown in the detailed description is based on the orientation or position shown in the figures of the accompanying drawings of the present disclosure, which is only to easily simplify the description of the present disclosure, but not indicated that the devices or elements of the present disclosure should have a particular orientation or should be designed and operated in a particular orientation. So the terms illustrated in the detail description are not by way of the limitation of the present disclosure.

In the description of the present disclosure, except where specifically otherwise illustrated or limited, the terms “connect” and “link” used herein should be understood in a broad sense. Such as, the meaning may be tight connection, removable connection, or integrated connection. The meaning may also be mechanical connection, electrical connection, direct connection or indirect connection through intermediaries, or internal connection within two elements. The meaning of the terms used herein may be understood by one of ordinary skill in the related art according to specific conditions of the present disclosure.

Furthermore, in the description of the present disclosure, the terms such as “first” and “second” shown in the specification are only used to describe, but not indicated that the elements of the present disclosure is important or represented the amount of the elements. That is, the features limited by the terms of “first” and “second” may explicitly or implicitly include one or more features.

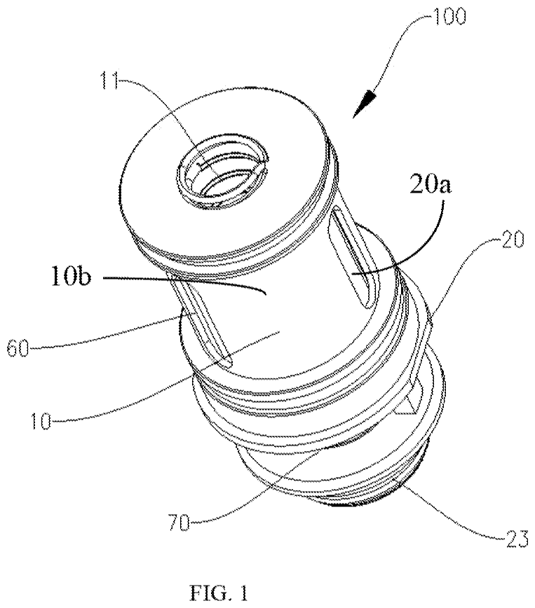

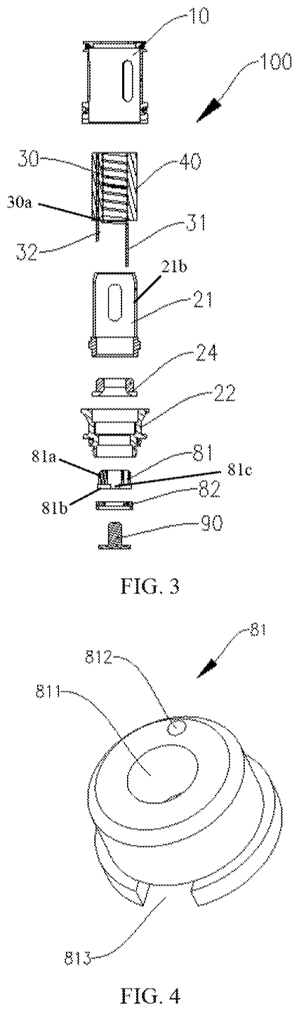

Referring to FIGS. 1-3, an atomization core 100 according to an exemplary embodiment of the present disclosure includes a housing 10, a frame 20, a heating wire 30 and an organic cotton 40. The frame 20 is an inner hollow structure to form a gas passage 50 therein. The housing 10 is covered around the frame 20 and includes an air outlet 11 formed at an upper portion 10a of the housing 10 to connect to the gas passage 50. The organic cotton 40 is sleeved around the heating wire 30 and received in the gas passage 50 of the frame 20. Each e-liquid inlet 60 is respectively formed at a sidewall 10b of the housing 10 and a sidewall 20a of the frame 20 to connect to the organic cotton 40. Two opposite first ends 30a of the heating wire 30 are respectively connected to a first polarity pin 31 and a second polarity pin 32. In the exemplary embodiment of the present disclosure, the first polarity pin 31 is a positive pin and the second polarity pin 32 is a negative pin. The tobacco liquid can enter the organic cotton 40 from the e-liquid inlet 60 and then can be atomized to be smoked by users under a heating action of the heating wire 30.

The frame 20 includes an air inlet 70 formed at the sidewall 20a of the bottom thereof for connecting to the gas passage 50, and a connecting portion 23 formed at a lower portion thereof near the air inlet 70 to directly connect to a battery assembly (not shown). External gas is passed from the air inlet 70 into the gas passage 50 of the atomization core 100, and then the fume from the gas passage 50 is discharged out from the air outlet 11.

The atomization core 100 further includes an insulating portion 80 mounted on a bottom 50a of the gas passage 50 and spaced from a bottom of the organic cotton 40, an electrode post 90 mounted on the insulating portion 80 and insulated from the frame 20. The first polarity pin 31 is extended along the bottom 50a of the gas passage 50 through the insulating portion 80 and connected to the electrode post 90, and the second polarity pin 32 is connected to an inner wall 20a of the frame 20. The insulating portion 80 and the electrode post 90 are respectively mounted on the bottom 50a of the frame 50 to seal the bottom 50a of the frame 20. At the same time, the insulating portion 80 and the organic cotton 40 are separated from each other so that gas can enter the gas passage 50 from the air inlet 70 of the sidewall 20a of the frame 20 and then be discharged out from the air outlet 11 along the gas passage 50, thereby avoiding the interference of the insulating portion 80 to the gas flow. The insulating portion 80 is provided for the electrode post 90 being insulated isolation from the frame 20 and the first polarity pin 31 is simultaneously connected to the electrode post 90 so that the contact between the first polarity pin 31 and the frame 20 can be prevented, thereby preventing short circuit from occurring therebetween. The electrode post 90 is arranged on a bottom of the atomization core 100, combined with the connecting portion 23 of the frame 20, and directly contacted with a battery of the battery assembly. In this way, a connection path between the first polarity pin 31 and the battery assembly can be avoided from needing electrode conductors on the base assembly provided for intermediate connection in the conventional technology so that the contact resistance is reduced. At the same time, it not only can avoid the resistance value jump after the thermal expansion and cold shrinkage deformation caused by an excessive machining error or assembly error due to too much middle conductive elements, but also can avoid the atomization core from becoming overheat because the contact resistance is too large or the resistance value jumps. Thus, the lifespan of the atomization core is improved.

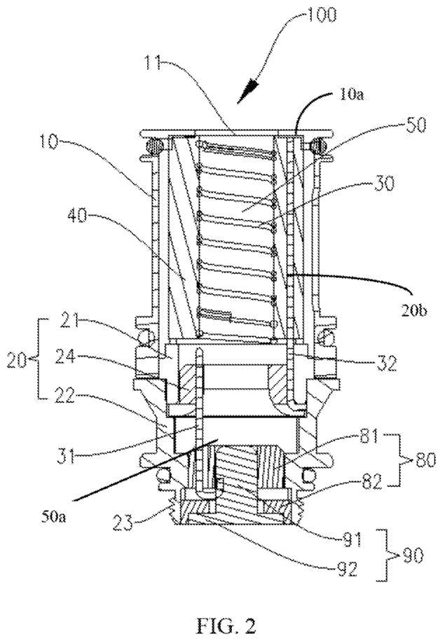

Specifically, referring to FIG. 2 and FIGS. 4-6, the insulating portion 80 includes an insulating gasket 81 and an electrode gasket 82 mounted in sequence at the bottom 50a of the gas passage 50. An upper portion 81a of the insulating gasket 81 is separated from the organic cotton 40 and a lower portion 81b of the insulating gasket 81 is attached to the electrode gasket 82. The insulating gasket 81 includes an installing hole 811 and a first through-hole 812 formed at interval along an axial direction thereof. The first through-hole 812 is connected with the installing hole 811 at a bottom 81c of the insulating gasket 81. The first through-hole 812 communicates with the installing hole 811 to facilitate a contact connection of the first polarity pin 31 with the electrode post 90.

The electrode gasket 82 includes a second through-hole 821 along its axis direction thereof. One end 821b of the second through-hole 82 is enlarged to form a sink hole 822 and the other opposite end 821a of the second through-hole 82 is connected to the installing hole 811. In an exemplary embodiment of the present disclosure, both the insulating gasket 81 and the electrode gasket 82 are made of insulation material.

The electrode post 90 includes a rod portion 91 passing through the second through-hole 821 and the installing hole 811, and a base portion 92 mounted on the sink hole 822. In this way, the electrode post 90 is installed inside the electrode gasket 82 and the insulating gasket 81 and insulated from an inner wall 20b of the frame 20.

The first polarity pin 31 is received in the first through-hole 812 and then connected to the electrode post 90 after bending its upper portion above an upper end 82a of the electrode gasket 82. The first polarity pin 31 is provided for passing through the first through-hole 812 for easy positioning and fixing the first polarity pin 31 and avoiding the contact between the first polarity pin 31 and the inner wall 20b of the frame 20. At the same time, the first polarity pin 31 is contacted with the electrode post 90 after bending it so that it can avoid the occurrence of jumping resistance caused by a poor contact between the first polarity pin 31 and the electrode post 90 after a thermal deformation of the insulating portion 80.

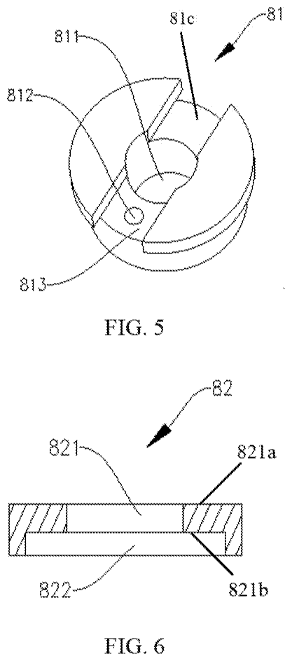

Referring to FIG. 4 and FIG. 5, the insulating gasket 81 further includes a connecting recess 813 formed at the bottom 81c thereof to connect to the first through-hole 812 and the installing hole 811. The first polarity pin 31 is connected to the rod portion 91 after bending it. The connecting recess 813 is provided for facilitating placing a bending part of the first polarity pin 31 in the connecting recess 813. At the same time, the bending part of the first polarity pin 31 can be avoided to interfere with a fit between the insulating gasket 81 and the electrode gasket 82.

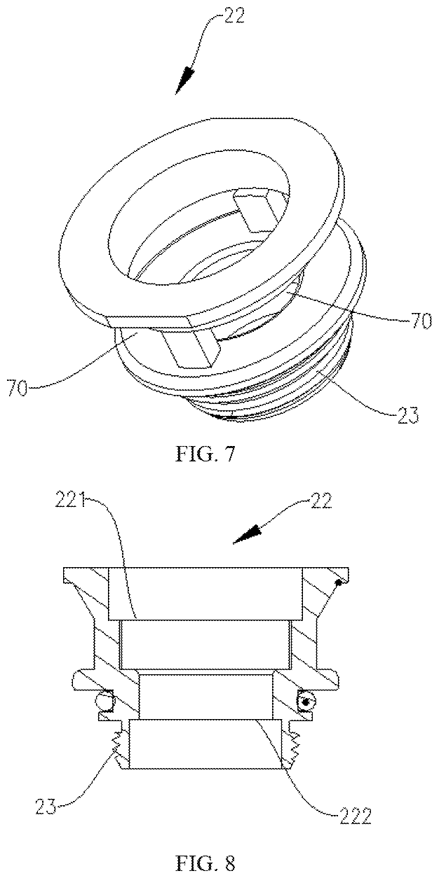

Referring to FIG. 2, FIG. 7 and FIG. 8, the frame 20 further includes a hollow upper frame 21, a hollow lower frame 22 and an insulating sleeve 24 formed between the upper and lower frames 21, 22, the gas passage 50 passing through the upper and lower frames 21, 22. The insulating sleeve 24 includes a connecting hole 241 formed in a middle portion thereof to connect the gas passage 50 of the upper and lower frames 21, 22, and a bottom portion of the upper frame 21 extending into the gas passage 50 of the lower frame 22. The housing 10 is sleeved on the upper frame 21, the e-liquid inlet 60 is arranged on the housing 10 and a sidewall of the upper frame 21. The air inlet 70 is arranged on a sidewall of the lower frame 22 and the connecting portion 23 is arranged on a lower end of the air inlet 70 of the lower frame. Both the organic cotton 40 and the heating wire 30 are received in the gas passage 50 of the upper frame 21. The insulating portion 80 and the electrode post 90 are respectively mounted on a bottom portion of the lower frame 22 to seal the bottom portion of the lower frame 22. The upper frame 21 and the lower frame 22 are provided for facilitating a separation between the organic cotton 40, the heating wire 30 and the insulating portion 80 and the electrode post 90, and simultaneously allowing the gas passage 50 to smoothly flow. In an exemplary embodiment of the present disclosure, the connecting portion 23 is a thread arranged in a ring direction on the outer wall of the lower frame 22. In this way, the screw structure is simple and easy to assemble or disassemble the atomization core 100 and the battery assembly.

The first polarity pin 31 passes through the connecting hole 241 of the insulating sleeve 24 and the second polarity pin 32 is pressed between an outer wall 24a of the insulating sleeve 24 and an inner wall 21b of the upper frame 21 to connect to the inner wall 21b of the upper frame 21. The insulating sleeve 24, on the one hand, can insulate the first polarity pin 31 from the inner wall 21b of the upper frame 21; on the other hand, can press the second polarity pin 32 against the inner wall 21b of die upper frame 21. In this way, short circuit between the first polarity pin 31 and the second polarity pin 32 can be avoided.

Specifically, referring to FIG. 2 and FIG. 8, the upper frame 21 further includes a first stepping surface 211 formed at an upper portion thereof, and a second stepping surface 212 formed at a lower portion thereof. An upper portion of the insulating sleeve 24 is inserted into the gas passage 50 of the upper frame 21, with a bottom surface of the insulating sleeve 24 abutting against the first stepping surface 211. The insulating gasket 81 is received in the gas passage 50 of the lower frame 22 and abutted against the second stepping surface 212, the bottom surface of the insulating sleeve 24 separated from a top surface of the insulating gasket 81. The first and second stepping surfaces 221, 222 are provided for respectively fixing the insulating sleeve 24 and the insulating gasket 81 at interval within the gas passage 50 of the lower frame 20 and allowing the gas passage 50 to smoothly flow, and simultaneously insulating the first polarity pin 31 from the inner wall 21b of the upper frame 21.

Preferably, referring to FIG. 9, the insulating sleeve 24 further includes an installing groove 242 formed at the outer wall of the bottom thereof, and the second polarity pin 32 pressed against the outer wall of the insulating sleeve 24 and the inner wall 21b of the upper frame 21 is contacted with an inner wall of the lower frame 22 after bending it in the installing groove 242. The installing groove 242 is provided for securely positioning the second polarity pin 32 to prevent the second polarity pin 32 from offsetting. At the same time, the contact between the second polarity pin 32 and the inner wall of the lower frame 22 after bending it can avoid the jumping resistance problem caused by a poor contact during using the second polarity pin 32.

Preferably, both the insulating portion 80 and the insulating sleeve 24 are made of high temperature resistant plastic material. The high temperature resistant plastic material can prevent a deformation of the insulating portion 80 and the insulating sleeve 24 after being heated so that the first and second polarity pins 31, 32 are poor contacted to lead a jumping resistance therebetween during use of the atomization core 100. In this way, it can further prevent the atomization core 100 from overheating due to the jumping resistance to improve the lifespan of the product.

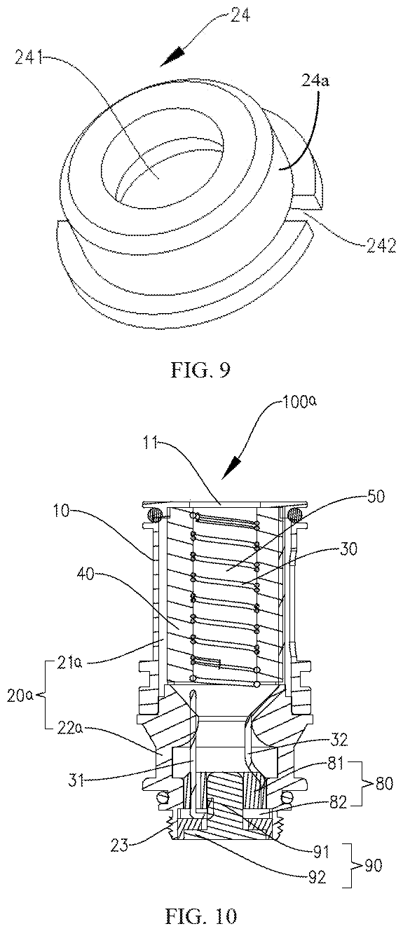

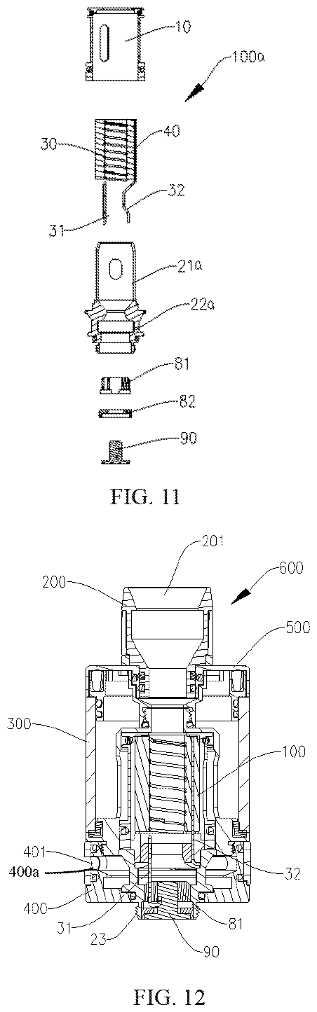

Referring to FIG. 10 and FIG. 11, an atomization core 100a according to a second exemplary embodiment of the present disclosure is provided. The difference between the two embodiments is the structure of the frame. In the second embodiment of the present disclosure, the frame 20a is a hollow integral structure and includes an upper frame portion 21a and a lower frame portion 22a. The housing 10 is sleeved on the upper frame portion 21a, the e-liquid inlet 60 is arranged on the housing 10 and a sidewall of the upper frame portion 21a. The air inlet 70 is arranged on a sidewall of the lower frame portion 22a and the connecting portion 23 is arranged on a lower end of the air inlet 70 of the lower frame 22a. The organic cotton 40 and the heating wire 30 are respectively received in the gas passage 50 of the upper frame portion 21a. The insulating portion 80 and the electrode post 90 are respectively mounted on a bottom portion of the lower frame portion 22a to seal the bottom portion of the lower frame portion 22a. The first polarity pin 31 passes through the lower frame portion 22a and connected to the electrode post 90, and the second polarity pin 32 passes through the lower frame portion 22a and pressed between an outer wall of the insulating gasket 81 and an inner wall of the lower frame portion 22a to connect to the inner wall of the lower frame portion 22a. The integrated frame 20 can minimize installation problems of the atomization core 100a.

Referring to FIG. 12, an atomizer 600 according to an exemplary embodiment of the present disclosure is provided. The atomizer 600 includes a hollow mouthpiece assembly 200, a cover member 500, an e-liquid storage assembly 300, a base assembly 400 and the above atomization core 100, 100a in sequential connection. FIG. 12 is shown the atomizer 600 with the atomization core 100 of the first embodiment. The housing 10 and one part of the frame 20 are received in the e-liquid storage assembly 300 and the other part of the frame 20 is received in the base assembly 400. The connecting portion 23 of the frame 20 is extended out of the base assembly 400 to connect to the battery assembly (not shown). The base assembly 400 includes a gas inlet 401 formed on a sidewall 400a thereof to connect to the air inlet 70 of the frame 20, and the mouthpiece assembly 200 includes a gas vent 201 so that gas can enter the gas passage 50 of the atomization core 100 from the gas inlet 401 of the base assembly 400 and then is discharged out from the gas vent 201 of the mouthpiece assembly 200. The atomizer 600 adopts the structure of the atomization core 100, 100a mentioned above. Therefore, the atomizer 600 can directly connect to the battery assembly by the connecting portion 23 of the atomization core 100, 100a and the electrode post 90, and the first polarity pin 31 can directly and electrically connect to a battery of the battery assembly by the electrode post 90, thereby the contact resistance can be reduced by reducing indirect contact of intermediate conductors. At the same time, no indirect conduction of intermediate conductors can also prevent the pulse of the resistance value. The atomization core can't become too hot because the contact resistance is too large or the resistance value jumps.

Each sealing ring (not labeled) is arranged at a connection joint of the housing 10 and the e-liquid storage assembly 300 of the atomization core 100, 100a of the present disclosure, and a connection joint of the frame 20 and base assembly 400 is provided to prevent e-liquid leakage.

The atomization core 100, 100a and the atomizer 600 of the present disclosure are provided the below configuration: the insulating portion 80 and the electrode post 90 are arranged on the bottom of the frame 20 so that the electrode post 90 is separated from the frame by the insulating portion 80; the organic cotton 40 and the heating wire 30 are received in the gas passage 50 of the frame 20, and the first polarity pin 31 of the heating wire 30 passes through the insulating portion 80 to connect to the electrode post 90 and the second polarity pin 32 is connected to the inner wall 21b of the frame 20. The connecting portion 23 is formed on the lower portion of the frame 20. In this way, the atomization core 100 can directly connect to the battery assembly by the connecting portion 23, the first polarity pin 31 of the heating wire 30 can directly and electrically connect to a battery of the battery assembly by the electrode post 90, thereby the contact resistance can be reduced by reducing an indirect contact of intermediate conductors. At the same time, no indirect conduction of intermediate conductors can also prevent the pulse of the resistance value. The atomization core can't become too hot because the contact resistance is too large or the resistance value jumps so that the lifespan of the atomization core can be improved.

Although the features and elements of the present disclosure are described as embodiments in particular combinations, each feature or element can be used alone or in other various combinations within the principles of the present disclosure to the full extent indicated by the broad general meaning of the terms in which the appended claims are expressed.

What is claimed is:

1. An atomization core, comprising:
a hollow frame comprising a gas passage formed along its axis direction thereof, an air inlet formed on a sidewall at a bottom thereof to connect with the gas passage, and a connecting portion formed at a lower portion thereof near the air inlet and directly connected to a battery assembly;
a housing covered on the frame and comprising an air outlet formed close to an upper end of the gas passage;
a heating wire comprising two opposite ends respectively connected to a first polarity pin and a second polarity pin;
an organic cotton surrounded around the heating wire and received in the gas passage;
an insulating portion mounted on a bottom of the gas passage and insulated isolation from a bottom of the organic cotton;
an electrode post mounted on the insulating portion and insulated from the frame; and
wherein each e-liquid inlet is respectively formed at a sidewall of the housing and a sidewall of the frame to connect to the organic cotton, and the first polarity pin is extended along the bottom of the gas passage through the insulating portion and connected to the electrode post, the second polarity pin is connected to an inner wall of the frame.

2. The atomization core as claimed in claim 1, wherein the insulating portion comprises an insulating gasket and an electrode gasket mounted in sequence on the bottom of the gas passage, with an upper portion of the insulating gasket separating from the organic cotton and a lower portion attaching to the electrode gasket;
the insulating gasket comprising an installing hole and a first through-hole formed at interval along an axial direction of the insulating gasket, the first through-hole connected with the installing hole at a bottom of the insulating gasket;
the electrode gasket comprising a second through-hole along its axis direction thereof, one end of the second through-hole enlarged to form a sink hole and the other opposite end of the second through-hole connected to the installing hole;
the electrode post comprising a rod portion passing through the second through-hole and the installing hole, and a base portion mounted on the sink hole; and
the first polarity pin received in the first through-hole and then connected to the electrode post after bending its upper portion above an upper end of the electrode gasket.

3. The atomization core as claimed in claim 2, wherein the insulating gasket further comprises a connecting recess formed at a bottom thereof to connect to the first through-hole and the installing hole, the first polarity pin connected to the rod portion after bending it.

4. The atomization core as claimed in claim 2, wherein the frame further comprises a hollow upper frame, a hollow lower frame and an insulating sleeve formed between the upper frame and the lower frame, the gas passage passing through the upper frame and the lower frame, the insulating sleeve comprising a connecting hole formed in a middle portion thereof to connect the gas passage of the upper and lower frames, a bottom portion of the upper frame extending into the gas passage of the lower frame, the housing sleeved on the upper frame, the e-liquid inlet arranged on the housing and a sidewall of the upper frame, the air inlet arranged on a sidewall of the lower frame and the connecting portion arranged on a lower end of the air inlet of the lower frame; both the organic cotton and the heating wire received in the gas passage of the upper frame; the insulating portion and the electrode post respectively mounted on a bottom portion of the lower frame to seal the bottom portion of the lower frame; the first polarity pin passing through the connecting hole of the insulating sleeve and the second polarity pin pressed between an outer wall of the insulating sleeve and an inner wall of the upper frame and connected to the inner wall of the upper frame.

5. The atomization core as claimed in claim 4, wherein the upper frame further comprises a first stepping surface formed at an upper portion thereof, and a second stepping surface formed at a lower portion thereof; an upper portion of the insulating sleeve inserting into the gas passage of the upper frame, with a bottom surface of the insulating sleeve abutting against the first stepping surface; the insulating gasket received in the gas passage of the lower frame and abutted against the second stepping surface, the bottom surface of the insulating sleeve separated from a top surface of the insulating gasket.

6. The atomization core as claimed in claim 5, wherein the insulating sleeve further comprises an installing groove formed at the outer wall of the bottom thereof, the second polarity pin pressed against the outer wall of the insulating sleeve and the inner wall of the upper frame is contacted with an inner wall of the lower frame after bending it in the installing groove.

7. The atomization core as claimed in claim 6, wherein both the insulating portion and the insulating sleeve are made of high temperature resistant plastic material.

8. The atomization core as claimed in claim 4, wherein the connecting portion is a thread arranged in a ring direction on the outer wall of the lower frame.

9. The atomization core as claimed in claim 2, wherein the frame is a hollow integral structure and comprises an upper frame portion and a lower frame portion, the housing sleeved on the upper frame portion, the e-liquid inlet arranged on the housing and a sidewall of the upper frame portion, the air inlet arranged on a sidewall of the lower frame portion and the connecting portion arranged on a lower end of the air inlet of the lower frame portion; the organic cotton and the heating wire respectively received in the gas passage of the upper frame portion; the insulating portion and the electrode post respectively mounted on a bottom portion of the lower frame portion to seal the bottom portion of the lower frame portion; the first polarity pin passing through the lower frame portion and connected to the electrode post, the second polarity pin passing through the lower frame portion and pressed between an outer wall of the insulating gasket and an inner wall of the lower frame portion and connected to the inner wall of the lower frame portion.

10. An atomizer comprising a hollow mouthpiece assembly, a cover member, an e-liquid storage assembly, a base assembly and an atomization core in sequential connection, the atomization core comprising:
a cylindrical hollow frame comprising a gas passage formed along its axis direction thereof, an air inlet formed on a sidewall at a bottom thereof to connect with the gas passage, and a connecting portion formed at a lower portion thereof near the air inlet and directly connected to a battery assembly;
a housing covered on the frame and comprising an air outlet formed close to an upper end of the gas passage;
a heating wire comprising two opposite ends respectively connected to a first polarity pin and a second polarity pin;
an organic cotton surrounded around the heating wire and received in the gas passage;
an insulating portion mounted on a bottom of the gas passage and insulated isolation from a bottom of the organic cotton;
an electrode post mounted on the insulating portion and insulated from the frame; and
wherein each e-liquid inlet respectively formed at a sidewall of the housing and a sidewall of the frame to connect to the organic cotton, and the first polarity pin extended along the bottom of the gas passage through the insulating portion and connected to the electrode post, the second polarity pin is connected to an inner wall of the frame;
the housing and one part of the frame received in the e-liquid storage assembly and the other part of the frame received in the base assembly, the connecting portion extending out of the base assembly to connect to the battery assembly; and wherein the base assembly comprises a gas inlet formed on a sidewall thereof to connect to the air inlet of the frame, and the mouthpiece assembly comprises a gas vent so that gas can enter the gas passage of the atomization core from the gas inlet of the base assembly and then is discharged out from the gas vent of the mouthpiece assembly.

11. The atomizer as claimed in claim 10, wherein the insulating portion comprises an insulating gasket and an electrode gasket mounted in sequence on the bottom of the gas passage, with an upper portion of the insulating gasket separating from the organic cotton and a lower portion attaching to the electrode gasket;
the insulating gasket comprising an installing hole and a first through-hole formed at interval along an axial direction of the insulating gasket, the first through-hole connected with the installing hole at a bottom of the insulating gasket;
the electrode gasket comprising a second through-hole along its axis direction thereof, one end of the second through-hole enlarged to form a sink hole and the other opposite end of the second through-hole connected to the installing hole;
the electrode post comprising a rod portion passing through the second through-hole and the installing hole, and a base portion mounted on the sink hole; and
the first polarity pin received in the first through-hole and then connected to the electrode post after bending its upper portion above an upper end of the electrode gasket.

12. The atomizer as claimed in claim 11, wherein the insulating gasket further comprises a connecting recess formed at a bottom thereof to connect to the first through-hole and the installing hole, the first polarity pin connected to the rod portion after bending it.

13. The atomizer as claimed in claim 11, wherein the frame further comprises a hollow upper frame, a hollow lower frame and an insulating sleeve formed between the upper frame and the lower frame, the gas passage passing through the upper frame and the lower frame, the insulating sleeve comprising a connecting hole formed in a middle portion thereof to connect the gas passage of the upper and lower frames, a bottom portion of the upper frame extending into the gas passage of the lower frame, the housing sleeved on the upper frame, the e-liquid inlet arranged on the housing and a sidewall of the upper frame, the air inlet arranged on a sidewall of the lower frame and the connecting portion arranged on a lower end of the air inlet of the lower frame; both the organic cotton and the heating wire received in the gas passage of the upper frame; the insulating portion and the electrode post respectively mounted on a bottom portion of the lower frame to seal the bottom portion of the lower frame; the first polarity pin passing through the connecting hole of the insulating sleeve and the second polarity pin pressed between an outer wall of the insulating sleeve and an inner wall of the upper frame and connected to the inner wall of the upper frame.

14. The atomizer as claimed in claim 13, wherein the upper frame further comprises a first stepping surface formed at an upper portion thereof, and a second stepping surface formed at a lower portion thereof; an upper portion of the insulating sleeve inserting into the gas passage of the upper frame, with a bottom surface of the insulating sleeve abutting against the first stepping surface; the insulating gasket received in the gas passage of the lower frame and abutted against the second stepping surface, the bottom surface of the insulating sleeve separated from a top surface of the insulating gasket.

15. The atomizer as claimed in claim 14, wherein the insulating sleeve further comprises an installing groove formed at the outer wall of the bottom thereof, the second polarity pin pressed against the outer wall of the insulating sleeve and the inner wall of the upper frame is contacted with an inner wall of the lower frame after bending it in the installing groove.

16. The atomizer as claimed in claim 15, wherein both the insulating portion and the insulating sleeve are made of high temperature resistant plastic material.

17. The atomizer as claimed in claim 13, wherein the connecting portion is a thread arranged in a ring direction on the outer wall of the lower frame.

18. The atomizer as claimed in claim 11, wherein the frame is a hollow integral structure and comprises an upper frame portion and a lower frame portion, the housing sleeved on the upper frame portion, the e-liquid inlet arranged on the housing and a sidewall of the upper frame portion, the air inlet arranged on a sidewall of the lower frame portion and the connecting portion arranged on a lower end of the air inlet of the lower frame portion; the organic cotton and the heating wire respectively received in the gas passage of the upper frame portion; the insulating portion and the electrode post respectively mounted on a bottom portion of the lower frame portion to seal the bottom portion of the lower frame portion; the first polarity pin passing through the lower frame portion and connected to the electrode post, the second polarity pin passing through the lower frame portion and pressed between an outer wall of the insulating gasket and an inner wall of the lower frame portion and connected to the inner wall of the lower frame portion.
